(19) United States
(12) Patent Application Publication
(10) Pub. No.: US 2025/0139776 A1
(43) Pub. Date: May 1, 2025

(54) SYSTEMS AND METHODS FOR CYTOLOGICAL ANALYSIS

(71) Applicant: Mars, Incorporated, McLean, VA (US)

(72) Inventors: Jan ANDREWS, Raleigh, NC (US); Sarah S. K. BEATTY, Gainesville, FL (US); Jennifer A. NEEL, Tucson, AZ (US); Rodrigo M. B. SANTANA, São José dos Campos (BR); Izzidin OAKES, Jersey City, NJ (US); Michael FITZKE, Oyten (DE)

(21) Appl. No.: 18/931,947

(22) Filed: Oct. 30, 2024

Related U.S. Application Data
(60) Provisional application No. 63/594,713, filed on Oct. 31, 2023.

(51) Int. Cl. G06T 7/00; G06T 7/11; G06V 10/44; G06V 10/82; G06V 20/69; G16B 15/10; G16H 10/60

(52) U.S. Cl. CPC G06T 7/0012; G06T 7/11; G06V 10/44; G06V 10/82; G06V 20/695; G16B 15/10; G16H 10/60; G06T 2207/20021; G06T 2207/20081; G06T 2207/20084; G06T 2207/30024; G06T 2207/30096; G06T 2207/30242; G06V 2201/03

(57) ABSTRACT

A computer-implemented method for cytological grading may include receiving first image data representing a biological sample, and parsing the received first image data into a plurality of tiles. Each of the plurality of tiles may represent a respective portion of the received first image data. The method may also include identifying, using a first trained machine learning model, at least one cytological feature for each of the plurality of tiles. The method may include determining, using a second trained machine learning model, at least one statistic based on the identified at least one cytological feature for each of the plurality of tiles, and outputting the determined at least one statistic.

### CROSS-REFERENCE TO RELATED APPLICATION(S)

This patent application claims the benefit of priority to U.S. Application No. 63/594,713, filed on Oct. 31, 2023, the entirety of which is incorporated herein by reference.

### TECHNICAL FIELD

Various embodiments of the present disclosure relate generally to systems and methods for cytological analysis. More specifically, the disclosure relates to systems and methods for cytologically analyzing and grading a sample (e.g., of a cutaneous mast cell tumor) using machine learning.

### BACKGROUND

Mast cell tumors (MCTs) are the most common cutaneous neoplasms in dogs. Traditionally, MCTs are histopathologically graded. For example, a pathologist may examine and assess tissue including an MCT once the tissue is surgically removed from a dog. However, demand for cytological grading (e.g., an evaluation of cells) of MCTs is increasing due to the availability of effective treatments for MCTs, such as intratumoural injection and electrochemotherapy. To perform cytological grading, a sample of an MCT may be collected from a dog using fine-needle aspiration, and the sample may be scanned by a Hamamatsu Scanner to generate a digital microscopic image of the sample. A pathologist may then cytologically grade the sample by examining, for example, the sizes of nuclei and/or the number of mitotic figures, depicted in the digital microscopic image. However, because the digital microscopic image may depict a high number of cells, it may be very time-consuming for the pathologist to examine, in detail, each of the depicted cells (or the entire digital microscopic image). Moreover, cytological grading of MCTs can be prone to inter-observer variability. Accordingly, there is a need for a more efficient and accurate means for cytologically analyzing and grading cutaneous MCTs in dogs, for example.

This disclosure is directed to addressing above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, embodiments are disclosed for cytological analysis and grading of a sample, using machine learning.

A computer-implemented method for cytological grading is disclosed. The method may include receiving first image data representing a biological sample, and parsing the received first image data into a plurality of tiles. Each of the plurality of tiles may represent a respective portion of the received first image data. The method may also include identifying, using a first trained machine learning model, at least one cytological feature for each of the plurality of tiles. In addition, the method may include determining, using a second trained machine learning model, at least one statistic based on the identified at least one cytological feature for each of the plurality of tiles, and outputting the determined at least one statistic.

A computer system for cytological grading is disclosed. The system may include at least one memory storing instructions, and at least one processor configured to execute the instructions to perform operations. Execution of the instructions may cause the at least one processor to receive first image data representing a biological sample, and parse the received first image data into a plurality of tiles. Each of the plurality of tiles may represent a respective portion of the received first image data. Execution of the instructions may further cause the at least one processor to identify, using a first trained machine learning model, at least one cytological feature for each of the plurality of tiles. Further, execution of the instructions may cause the at least one processor to determine, using a second trained machine learning model, at least one statistic based on the identified at least one cytological feature for each of the plurality of tiles, and output the determined at least one statistic.

A non-transitory computer-readable medium storing instructions is disclosed. When the instructions are executed by at least one processor, the instructions cause the at least one processor to perform operations for cytological grading. The operations may include receiving first image data representing a biological sample, and parsing the received first image data into a plurality of tiles. Each of the plurality of tiles may represent a respective portion of the received first image data. The operations may also include identifying, using a first trained machine learning model, at least one cytological feature for each of the plurality of tiles. In addition, the operations may include determining, using a second trained machine learning model, at least one statistic based on the identified at least one cytological feature for each of the plurality of tiles, and outputting the determined at least one statistic.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a flowchart illustrating an exemplary process for cytologically analyzing and grading a sample of a cutaneous MCT, according to one or more embodiments.

FIG. 2 depicts an exemplary block diagram of a system and network for cytologically analyzing and grading a sample, according to one or more embodiments.

FIG. 3 depicts a flowchart illustrating an exemplary process for cytological grading, according to one or more embodiments.

FIG. 4 depicts a flowchart illustrating an exemplary process for cytological grading, according to one or more embodiments.

FIG. 5A depicts an example tile of a plurality of tiles, according to one or more embodiments.

FIG. 5B depicts an example tile of a plurality of tiles, according to one or more embodiments.

FIG. 6 depicts an example system that may execute the techniques presented herein.

### DETAILED DESCRIPTION

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” “includes,” “including,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The term “veterinarian” may include, for example, without limitation, any person, organization, and/or collection of persons that provides medical care to non-human animals. For example, a “veterinarian” may include a veterinary technician, a veterinary personnel, and a veterinarian practitioner.

The terms “canine” and “dog” may include, for example, without limitation, recognized dog breeds (some of which may be further subdivided). For example, the recognized dog breeds may include Afghan hound, Airedale, akita, Alaskan malamute, basset hound, beagle, Belgian shepherd, bloodhound, border collie, border terrier, borzoi, boxer, bulldog, bull terrier, cairn terrier, Chihuahua, chow, cocker spaniel, collie, corgi, dachshund, Dalmatian, Doberman, English setter, fox terrier, German shepherd, golden retriever, great dane, greyhound, griffon bruxellois, Irish setter, Irish wolfhound, King Charles spaniel, Labrador retriever, Lhasa apso, mastiff, Newfoundland, old English sheepdog, Papillion, Pekingese, pointer, Pomeranian, poodle, pug, Rottweiler, St. Bernard, saluki, Samoyed, schnauzer, Scottish terrier, Shetland sheepdog, shih Tzu, Siberian husky, Skye terrier, springer spaniel, West Highland terrier, Yorkshire terrier, etc.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as a neural network(s), convolutional neural network(s), regional convolutional neural network(s), mask regional convolutional neural network(s), deformable detection transformer(s), linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. Any suitable type of training may be used, e.g., stochastic, gradient boosted, random seeded, recursive, epoch or batch-based, etc.

The term “grade” may include, for example, a measure or assessment of how abnormal cancer cells appear when viewed in a digital microscope image or under a microscope. In cytology, a grade may be determined by evaluating the appearance and/or behavior of cells. In histopathology, a grade may be determined by evaluating the structure and function of tissue.

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

In the following description, embodiments will be described with reference to the accompanying drawings. As will be discussed in more detail below, various embodiments, methods, systems, and computer readable media for cytologically analyzing and grading a sample (or specimen) are disclosed.

In an exemplary use case, a sample of a cutaneous MCT in a dog may be collected from the dog using fine-needle aspiration. The sample may be stained and then-scanned by a digital scanner (e.g., a Hamamatsu scanner) to generate a digital microscopic image of the stained sample. In some embodiments, the digital microscopic image may be parsed (or divided) into tiles, where each tile represents a respective portion of the digital microscopic image. The tiles may be processed by a first trained machine learning model that includes one or more trained sub-models, where each of the trained sub-models is configured to identify (or detect) a respective cytological feature depicted in the tiles. For example, in some embodiments, a first trained machine learning sub-model may be used to identify mast cells in each of the tiles, a second trained machine learning sub-model may be used to identify mitotic figures in each of the tiles, and a third trained machine learning sub-model may be used to identify mast cell nuclei in each of the tiles, and a fourth trained machine learning sub-model may be used to identify multinucleated cells in each of the tiles. Further, in some embodiments, each of the detected cytological features may be statistically analyzed and graded using a second trained machine learning model. For example, the second trained machine learning model may determine a first ratio of the total number of mitotic figures identified in the tiles relative to the total number of mast cells identified in the tiles. In addition, or in the alternative, the second trained machine learning model may determine a second ratio of the total number of multinucleated cells identified in the tiles relative to the total number of mast cells identified in the tiles. The second trained machine learning model may also determine a cytological grade (or assessment) of the sample based on the first ratio and/or the second ratio. In addition, the second trained machine learning model may determine whether a correlation exists between the cytological grade and a histopathological grade of the sample.

As described above, existing techniques for examining an entire digital microscopic image are often inefficient, and traditional cytological grading systems can be prone to inter-observer variability. However, aspects of the present disclosure provide processes for more efficiently analyzing an entire digital microscopic image, and more accurately grading a specimen depicted in the image. Moreover, embodiments described herein provide for a more detailed and complete cytological analysis of a specimen, and may thereby support improved clinical decision-making.

While the example above involves a sample of a cutaneous MCT in a dog, it should be understood that techniques according to this disclosure may be adapted to any suitable sample (e.g., cells associated with cancer, an infectious agent, or other disease or condition) retrieved from a canine or non-canine (e.g., a human, cat, bird, etc.) patient. Further, it should also be understood that the example above is illustrative only. The techniques and technologies of this disclosure may be adapted to any suitable application.

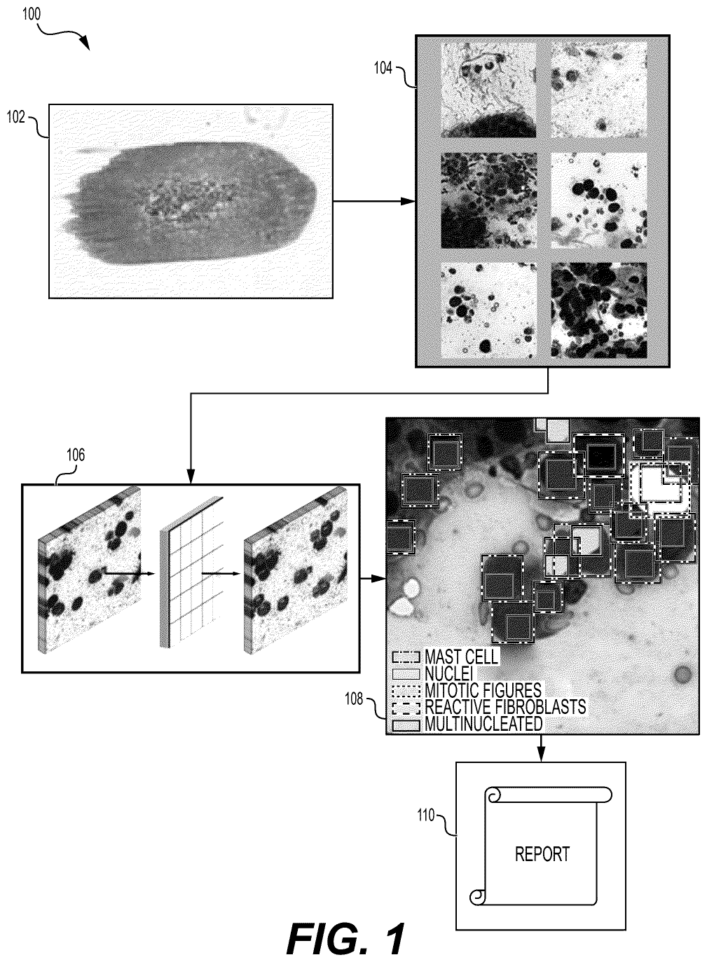

FIG. 1 depicts a flowchart illustrating an exemplary process 100 for cytologically analyzing and grading a stained sample of a cutaneous MCT, according to one or more embodiments. In some aspects, the sample may be retrieved from a canine using fine-needle aspiration, and then placed on a glass slide and stained. As shown in FIG. 1, the process 100 may include generating a whole slide image (or a digital microscopic image) of the stained sample using a digital scanner, such as a Hamamatsu NanoZoomer® S360 digital slide scanner, at step 102. Aspects of the present disclosure recognize that the whole slide image represents a Hamamatsu NanoZoomer Digital Pathology Image (NDPI) file, with a high pixel resolution (e.g., 200,000 pixels×100,000 pixels). However, in some embodiments, the whole slide image may be a different (or non-NDPI) type of file. In some aspects, the whole slide image may depict a large number of cells.

In some embodiments, the process 100 may include parsing (or dividing) the whole slide image into multiple tiles using a computing device (e.g., a workstation, desktop computer, laptop, etc.), at step 104. In some aspects, each of the tiles may depict a respective (or different) portion of the whole slide image. While FIG. 1 shows only six tiles for simplicity, the whole slide image may be parsed into any number of tiles (e.g., thousands of tiles) suitable for analysis by one or more machine learning models. In some aspects, dividing a whole slide image into multiple tiles may facilitate training of a machine learning model.

The process 100 may further include processing each of the tiles of the whole slide image using a first trained machine learning model, at step 106. In some embodiments, the first trained machine learning model may include one or more trained machine learning sub-models (also referred to herein as “sub-models”), where each of the one or more sub-models is configured to identify (or detect) a respective cytological feature. For example, the first trained machine learning model may include a first sub-model (e.g., a mask regional convolutional neural network model) configured to detect the number and/or location of each mast cell (if any) depicted in each of the tiles. The first trained machine learning model may also include a second sub-model (e.g., a mask regional convolutional neural network model) configured to detect the number, location, and/or size, of each mast cell nuclei (if any) depicted in each of the tiles. The first trained machine learning model may further include a third sub-model (e.g., a mask regional convolutional neural network model) configured to detect the number and/or location of mitotic figures (if any) depicted in each of the tiles. Additionally, the first trained machine learning model may include a fourth sub-model (e.g., a deformable detection transformer model) configured to detect the number and/or location of multinucleated cells (if any) in each of the tiles. In some aspects, each of the first, second, third, and fourth sub-models may be configured to not only identify a respective cytological feature, but also annotate (e.g., using bounding boxes and/or various colors) each of the identified features in the tiles. Furthermore, using one or more of the first, second, third, and fourth sub-models (or a respective mask regional convolutional neural network model to identify each of mast cells, mast cell nuclei, and mitotic figures, and a deformable detection model to identify multinucleated cells) may facilitate accurate cytological grading of the MCT sample (e.g., cytological grading that correlates with a known histopathologic grade of the MCT sample, as further described below). In some embodiments, in addition (or as an alternative) to the first, second, third, and fourth sub-models, the first trained machine learning model may include one or more sub-models configured to identify and annotate other cytologic features such as reactive fibroblasts and pleomorphism.

In some embodiments, the first trained machine learning model may output a digital image (or digital image data) in which one or more of the identified cytological features is annotated, as shown at step 106. More specifically, the first trained machine learning model may output a digital image in which identified mast cells, nuclei, mitotic figures, multinucleated cells, and/or other cytologic features are indicated using bounding boxes and/or various colors, along with a corresponding legend. In some aspects, the whole slide image (or one or more tiles of the whole slide image) may correspond, or be mapped, to the digital image output from the first trained machine learning model. In some embodiments, the first trained machine learning model may also output numerical data representing, for example, the number, size, and/or location (within a given tile and/or the whole slide image) of one or more of the identified cytological features. For example, the first trained machine learning model may output the number, size and location (within a given tile and the whole slide image) of identified mast cells, mast cell nuclei, mitotic figures, and multinucleated cells.

In some embodiments, a second trained machine learning model (e.g., a logistic regression model) may receive, as input, the numerical data (and optionally the annotated digital image data) output by the first trained machine learning model. The second trained machine learning model may be configured to determine one or more statistics based on the received numerical data. For example, the second trained machine learning model may be configured to determine the total number of mast cells, the total number of mitotic figures, the total number of mast cell nuclei, and the total number of multinucleated cells, identified in the whole slide image by the first machine learning model. The second trained machine learning model may also be configured to determine a first ratio of the total number of mitotic figures identified in the whole slide image to the total number of mast cells identified in the whole slide image. The second trained machine learning model may also be configured to determine a second ratio of the total number of multinucleated cells identified in the whole slide image to the total number of mast cells identified in the whole slide image. Further, the second trained machine learning model may be configured to determine a size distribution of multinucleation for the whole slide image. Moreover, the second trained machine learning model may be configured to determine a cytologic grade of the cutaneous MCT based on, for example, the first ratio and/or the second ratio.

In some embodiments, the second trained machine learning model may be configured to determine whether there is a correlation between the cytologic grade of the cutaneous MCT in the sample and a known histopathological grade of the cutaneous MCT. The known histopathological grade may be based on, for example, the Kiupel grading system and/or the Patnaik grading system. In some embodiments, the second trained machine learning model may be configured to determine whether there is a correlation between the cytologic grade of the cutaneous MCT in the sample and a known clinical outcome.

As shown in FIG. 1, the process 100 may include outputting a report at step 110. The report may include, for example, the cytologic grade and/or one or more statistics, output by the second trained machine learning model. The report may also, or in the alternative, include an annotated image and/or numerical data output by the first trained machine learning model. In some embodiments, one or more of the cytologic grade, the statistic(s), the numerical data and/or the annotated image may be input to a third trained machine learning model (e.g., a generative machine learning model) configured to generate a customized report based on the received cytologic grade, statistic(s), numerical data, annotated image, and/or other received data (e.g., data representing an intended reader of the report). For example, the third trained machine learning model may be configured to generate a report tailored to an intended reader such as a pathologist, a veterinarian, or a layperson). In some embodiments, the third trained machine learning model may be configured to use one or more templates to generate a customized report. While not shown in FIG. 1, in some embodiments, the process 100 may include inputting data representing, for example, blood test results and/or x-rays, into one or more machine learning models. The one or more machine learning models may be configured to identify features in the inputted data, statistically analyze the identified features, and determine a grade in a manner similar to that described above.

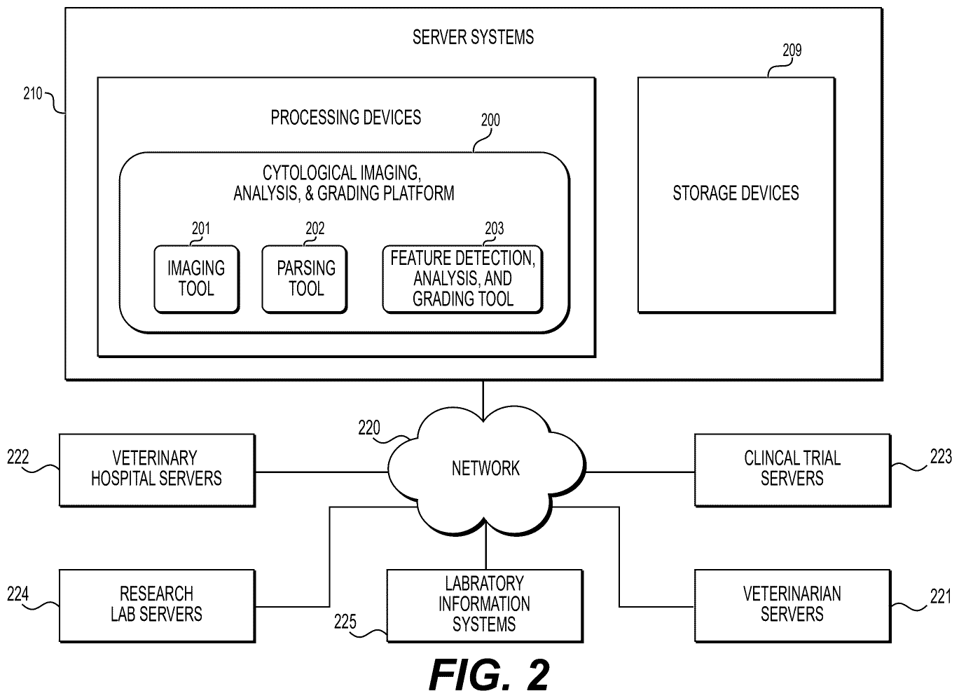

FIG. 2 depicts an exemplary block diagram of a system and network for cytologically analyzing and grading a sample, according to one or more embodiments. Specifically, FIG. 2 illustrates an electronic network 220 that may be connected to servers at hospitals (e.g., veterinary hospitals), laboratories, and/or doctors' offices (e.g., veterinarians' offices), etc. For example, each of veterinarian servers 221, veterinary hospital servers 222, clinical trial servers 223, research lab servers 224, and/or laboratory information systems 225, etc., may be connected to the electronic network 220, such as the Internet, through one or more computers, servers, and/or handheld mobile devices. According to an exemplary embodiment of the present application, the electronic network 220 may also be connected to server systems 210, which may include one or more storage devices 209 for storing images (e.g., whole slide images, etc.) and data (e.g., histopathological grades, etc.) received from at least one of the veterinarian servers 221, the veterinary hospital servers 222, the clinical trial servers 223, the research lab servers 224, and/or the laboratory information systems 225. The server systems 210 may also include processing devices such as workstations, computers, laptops, or other electronic devices. The processing devices may be configured to process images and data stored in the storage devices 209, using a cytological imaging, analysis, and grading platform 200 (also referred to herein as the “platform 200”).

As shown in FIG. 2, the platform 200 may include an imaging tool 201, a parsing tool 202, and a feature detection, analysis, and grading tool 203. In some embodiments, the imaging tool 201 may include at least one digital scanner (e.g., a Hamamatsu NanoZoomer® S360 digital slide scanner) configured to generate a whole slide image, as described above with respect to step 102 of FIG. 1. Further, in some embodiments, one or more of the veterinary hospitals, laboratories, veterinarians' offices, etc. associated with the servers 221-225 may include the imaging tool 201. The parsing tool 202 may be a software module configured to parse (or divide) a whole slide image generated by the imaging tool 201, as described above with respect to step 104 of FIG. 1. The feature detection, analysis, and grading tool 203 may include, for example, the first and second trained machine learning models discussed above with reference to FIG. 1, and be configured to perform steps 106, 108, and 110 of FIG. 1.

In some embodiments, one or more of the veterinary hospital, laboratories, veterinarians' offices, and other entities associated with the servers 221-225 may use the imaging tool 201 to generate a whole slide image of a cytological sample (e.g., a sample of a cutaneous MCT collected from a canine patient). Further, in some embodiments, a whole slide image may be transmitted by one of the servers 221-225, via the electronic network 220, to the parsing tool 202. In some other embodiments, the whole slide image may be transmitted directly from the imaging tool 201 to the parsing tool 202. In some aspects, each of the servers 221-225 may be configured to transmit, via the electronic network 220, a histopathological grade corresponding to a sample depicted in a whole slide image, to the feature detection, analysis, and grading tool 203. In addition, each of the servers 221-225 may be configured to receive, from the feature detection, analysis, and grading tool 203 and via the electronic network 220, a report that includes, for example, an analysis and grade associated a whole slide image. The report may be read by a pathologist, veterinarian, layperson, or other individual.

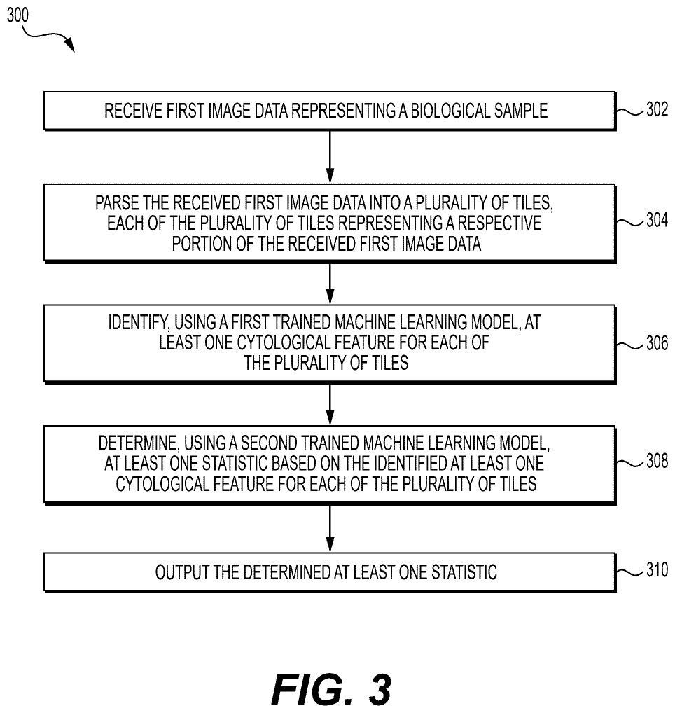

FIG. 3 depicts a flowchart illustrating an exemplary process 300 for cytological grading, according to one or more embodiments. In some aspects, the process 300 may be performed using the parsing tool 202 and the feature detection, analysis, and grading tool 203, discussed above with reference to FIG. 2. As shown in FIG. 3, the process 300 may include receiving first image data representing a biological sample (step 302). In some embodiments, the first image data may be a digital microscopic image (e.g., a whole slide image), and the biological sample may be a stained biological sample of, for example, an MCT collected from a dog. The process 300 may include parsing the received first image data into a plurality of tiles, where each of the plurality of tiles represents a respective portion of the received first image data (step 304). The method 300 may further include identifying, using a first trained machine learning model, at least one cytological feature for each of the plurality of tiles (step 306). The method 300 may also include determining, using a second trained machine learning model, at least one statistic based on the identified at least one cytological feature for each of the plurality of tiles (308). In some embodiments, the at least one statistic may be associated with a tumor (e.g., an MCT) or an infectious agent (e.g., a bacteria, a virus, or a fungus).

The method 300 may include outputting the determined at least one statistic (310). In some embodiments, the outputting of step 310 may include outputting a report that includes the at least one statistic. Further, in some embodiments, the process 300 may include generating, using a third trained machine learning model, the report based on the determined at least one statistic. The third trained machine learning model may include a generative machine learning model configured to generate the report based on data representing an intended reader of the report. In some embodiments, the process 300 may include outputting, by the first trained machine learning model and/or the second trained machine learning model, second image data representing an annotated image.

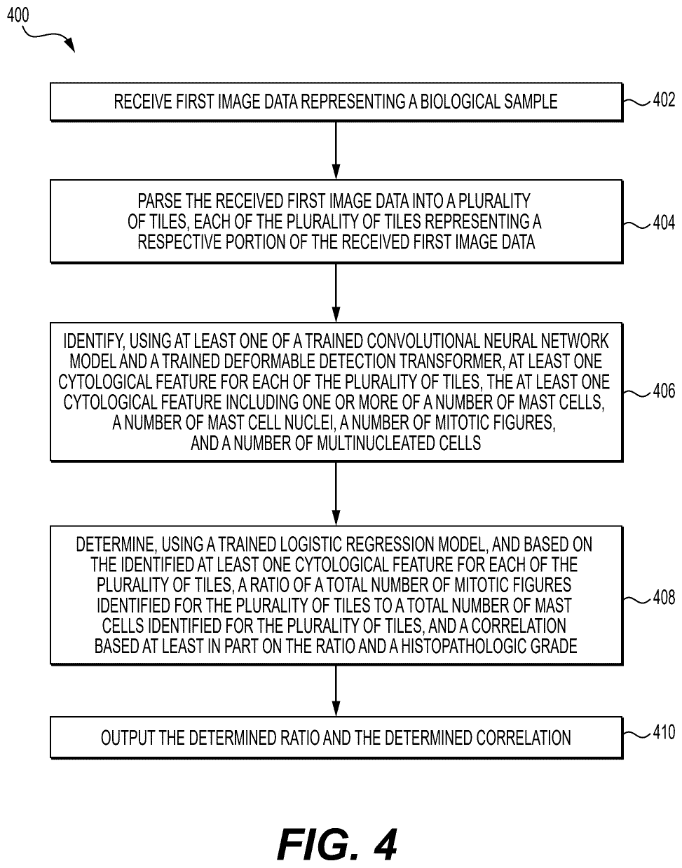

FIG. 4 depicts a flowchart illustrating an exemplary process 400 for cytological grading, according to one or more embodiments. Process 400 may be an embodiment of the process 300 of FIG. 3. In some aspects, the process 400 may be performed by the parsing tool 202 and the feature detection, analysis, and grading tool 203, discussed above with reference to FIG. 2.

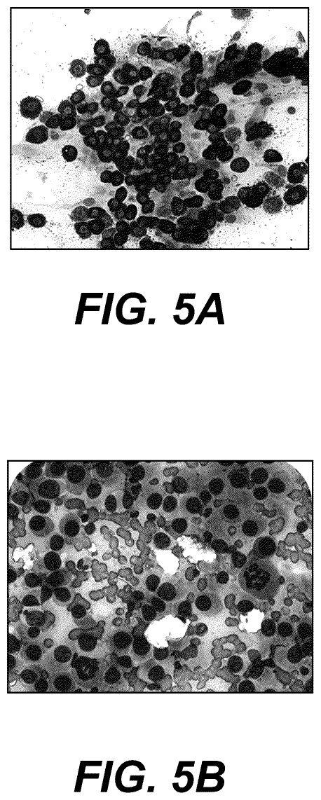

As shown in FIG. 4, the process 400 may include receiving first image data representing a biological sample (step 402). In some embodiments, the first image data may represent a digital microscopic image (e.g., the whole slide image discussed above with reference to step 102 of FIG. 1), and the biological sample may be a stained biological sample of, for example, an MCT collected from a dog. The process 400 may include parsing the received first image data into a plurality of tiles, where each of the plurality of tiles represents a respective portion of the received first image data (step 404). FIG. 5A depicts a first example tile of the plurality of tiles (where the first example tile shows low-grade cytological features of a cutaneous mast cell tumor from a dog). FIG. 5B depicts a second example tile of the plurality of tiles (where the second example tile shows high-grade cytological features of a cutaneous mast cell tumor from a dog). In some aspects, the received first image data may be parsed into any number of tiles suitable for processing using one or more machine learning models.

The process 400 may further include identifying, using at least one of a trained convolutional neural network model and a trained deformable detection transformer, at least one cytological feature for each of the plurality of tiles, the at least one cytological feature including one or more of a number of mast cells, a number of mast cell nuclei, a number of mitotic figures, and a number of multinucleated cells (step 406). In some embodiments, the at least one cytological feature may also, or in the alternative, include the location(s) of one or more mast cells, mast cell nuclei, mitotic figures, and multinucleated cells, with respect to a tile and/or the first image data (e.g., a whole slide image). The at least one cytological feature may also, or in the alternative, include the size of one or more mast cells, mast cell nuclei, mitotic figures, and multinucleated cells.

In some embodiments, the trained convolutional neural network model may include a first trained mask regional convolutional neural network sub-model configured to identify a total number of mast cells for each of the plurality of tiles. The trained convolutional neural network model may also, or in the alternative, include a second trained mask regional convolutional neural network sub-model configured to identify a total number of mitotic figures for each of the plurality of tiles. The trained convolutional neural network model may also, or in the alternative, include a third trained mask regional convolutional neural network sub-model configured to identify a total number of mast cell nuclei for each of the plurality of tiles. The trained deformable detection transformer may be configured to identify a total number of multinucleated cells for each of the plurality of tiles.

In some embodiments, the process 400 may further include determining, using a trained logistic regression model, and based on the identified at least one cytological feature for each of the plurality of tiles, a ratio of a total number of mitotic figures identified for the plurality of tiles to a total number of mast cells identified for the plurality of tiles, and a correlation based at least in part on the ratio and a histopathologic grade (step 408). In some embodiments, the process 400 may further, or in the alternative, include determining, using the trained logistic regression model, and based on the identified at least one cytological feature for each of the plurality of tiles, a ratio of a total number of multinucleated cells identified for the plurality of tiles to a total number of mast cells identified for the plurality of tiles, and a correlation based at least in part on the ratio and a histopathologic grade. In some embodiments, the process 400 may further include outputting the determined ratio(s) and the determined correlation(s) (410).

In general, any process or operation discussed in this disclosure that is understood to be computer-implementable, such as the process illustrated in FIG. 3 or FIG. 4, may be performed by one or more processors of a computer system. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices. One or more processors of a computer system may be included in a single computing device or distributed among a plurality of computing devices. A memory of the computer system may include the respective memory of each computing device of the plurality of computing devices.

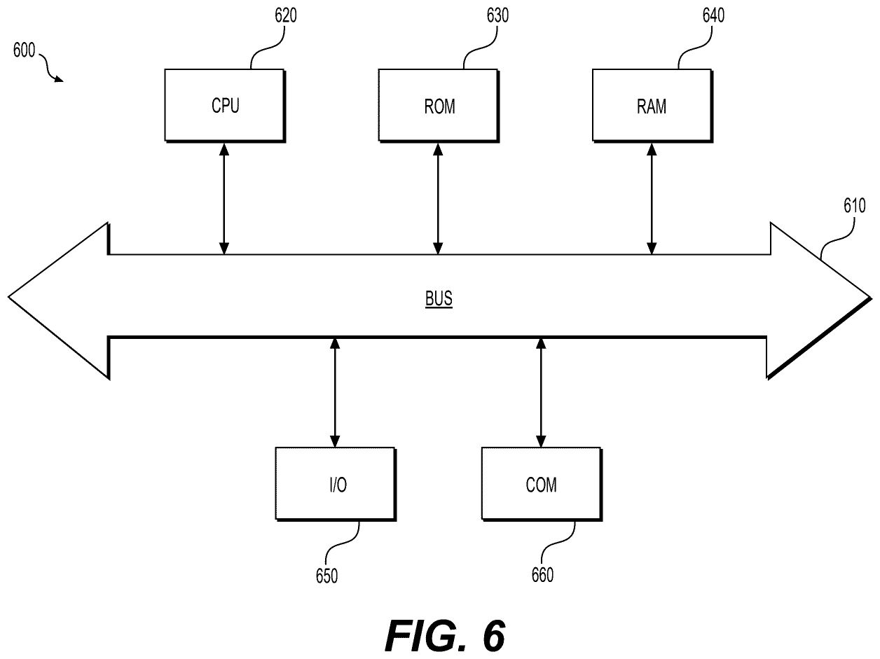

FIG. 6 is a simplified functional block diagram of a device 600 that may be configured to execute the method 300 and/or 400 of FIGS. 3 and 4, respectively, according to exemplary embodiments of the present disclosure. Put differently, the device 600 may be configured as the parsing tool 202 and/or the feature detection, analysis and grading tool 203 of FIG. 2, according to exemplary embodiments of this disclosure. As shown in FIG. 6, the device 600 may include a central processing unit (CPU) 620. The CPU 620 may be any type of processor device including, for example, any type of special purpose or a general-purpose microprocessor device. As will be appreciated by persons skilled in the relevant art, the CPU 620 also may be a single processor in a multi-core/multiprocessor system, such system operating alone, or in a cluster of computing devices operating in a cluster or server farm. The CPU 620 may be connected to a data communication infrastructure 610, for example, a bus, message queue, network, or multi-core message-passing scheme.

The device 600 also may include a main memory 640, for example, random access memory (RAM), and also may include a secondary memory 630. Secondary memory 630, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, the secondary memory 630 may include other similar means for allowing computer programs or other instructions to be loaded into the device 600. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to the device 600.

The device 600 also may include a communications interface (“COM”) 660. The communications interface 660 allows software and data to be transferred between the device 600 and external devices. The communications interface 660 may include a modem, a network interface (such as an Ethernet card), a communications port, a PCMCIA slot and card, or the like. Software and data transferred via the communications interface 660 may be in the form of signals, which may be electronic, electromagnetic, optical, or other signals capable of being received by the communications interface 660. These signals may be provided to communications interface 660 via a communications path of the device 600, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. The device 600 also may include input and output ports 650 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically can be grouped together to perform a function or group of related functions. Like reference numerals are generally intended to refer to the same or similar components. Components and modules can be implemented in software, hardware, or a combination of software and hardware.

The tools, modules, and functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other embodiments of the invention will be apparent to those skilled in the art from consideration of the specification and practice of the invention disclosed herein. It is intended that the specification and examples be considered as exemplary only.

What is claimed is:

1. A computer-implemented method for cytological grading, comprising:
receiving first image data representing a biological sample;
parsing the received first image data into a plurality of tiles, each of the plurality of tiles representing a respective portion of the received first image data;
identifying, using a first trained machine learning model, at least one cytological feature for each of the plurality of tiles;
determining, using a second trained machine learning model, at least one statistic based on the identified at least one cytological feature for each of the plurality of tiles; and
outputting the determined at least one statistic.

2. The computer-implemented method of claim 1, wherein identifying the at least one cytological feature for each of the plurality of tiles includes identifying at least one of a number of mast cells, a number of mast cell nuclei, a number of mitotic figures, and a number of multinucleated cells.

3. The computer-implemented method of claim 1, wherein the determined at least one statistic includes a ratio of a total number of mitotic figures identified for the plurality of tiles to a total number of mast cells identified for the plurality of tiles.

4. The computer-implemented method of claim 3, wherein the determined at least one statistic further includes a correlation based at least in part on the ratio and a histopathologic grade.

5. The computer-implemented method of claim 1, wherein the determined at least one statistic includes a ratio of a total number of multinucleated cells identified for the plurality of tiles to a total number of mast cells identified for the plurality of tiles.

6. The computer-implemented method of claim 5, wherein the determined at least one statistic further includes a correlation based at least in part on the ratio and a histopathologic grade.

7. The computer-implemented method of claim 1, wherein the first trained machine learning model includes at least one of a convolutional neural network sub-model and a deformable detection transformer sub-model, and wherein the second trained machine learning model includes a logistic regression model.

8. The computer-implemented method of claim 1, wherein the first trained machine learning model includes a mask regional convolutional neural network sub-model configured to identify a total number of mast cells for each of the plurality of tiles.

9. The computer-implemented method of claim 1, wherein the first trained machine learning model includes a mask regional convolutional neural network sub-model configured to identify a total number of mast cell nuclei for each of the plurality of tiles.

10. The computer-implemented method of claim 1, wherein the first trained machine learning model includes a mask regional convolutional neural network sub-model configured to identify a total number of mitotic figures for each of the plurality of tiles.

11. The computer-implemented method of claim 1, wherein the first trained machine learning model includes a deformable detection transformer sub-model configured to identify a total number of multinucleated cells for each of the plurality of tiles.

12. The computer-implemented method of claim 1, wherein the determined at least one statistic is associated with a tumor.

13. The computer-implemented method of claim 1, wherein the determined at least one statistic is associated with an infectious agent.

14. The computer-implemented method of claim 1, further comprising:
outputting second image data representing an annotated image.

15. The computer-implemented method of claim 1, wherein outputting the determined at least one statistic includes outputting a report.

16. The computer-implemented method of claim 1, further comprising:
generating, using a third trained machine learning model, a report based on the determined at least one statistic, wherein outputting the determined at least one statistic includes outputting the report.

17. The computer-implemented method of claim 16, wherein the third trained machine learning model includes a generative machine learning model configured to generate the report based on data representing an intended reader of the report.

18. A computer system for cytological grading, the computer system comprising:
at least one memory storing instructions; and
at least one processor configured to execute the instructions to perform operations comprising:
receiving first image data representing a biological sample;
parsing the received first image data into a plurality of tiles, each of the plurality of tiles representing a respective portion of the received first image data;
identifying, using a first trained machine learning model, at least one cytological feature for each of the plurality of tiles;
determining, using a second trained machine learning model, at least one statistic based on the identified at least one cytological feature for each of the plurality of tiles; and
outputting the determined at least one statistic.

19. The computer system of claim 18, wherein identifying the at least one cytological feature for each of the plurality of tiles includes identifying at least one of a number of mast cells, a number of mast cell nuclei, a number of mitotic figures, and a number of multinucleated cells.

20. A non-transitory computer-readable medium storing instructions that, when executed by at least one processor, cause the processor to perform operations for cytological grading, the operations comprising:
receiving first image data representing a biological sample;
parsing the received first image data into a plurality of tiles, each of the plurality of tiles representing a respective portion of the received first image data;
identifying, using a first trained machine learning model, at least one cytological feature for each of the plurality of tiles;
determining, using a second trained machine learning model, at least one statistic based on the identified at least one cytological feature for each of the plurality of tiles; and
outputting the determined at least one statistic.
